(19) United States
(12) Patent Application Publication
(10) Pub. No.: US 2025/0019692 A1
(43) Pub. Date: Jan. 16, 2025

(54) METHODS FOR THE DESIGN AND OPTIMISATION OF CHIMERIC ANTIGEN RECEPTORS (CARS)

(71) Applicant: CODING BIO LIMITED, Oxford (GB)

(72) Inventors: Simon BORNSCHEIN, Oxford (GB); Youlia LAMPI, Oxford (GB); Jonathan LEES, Oxford (GB)

(21) Appl. No.: 18/710,317

(22) PCT Filed: Nov. 16, 2022
(86) PCT No.: PCT/GB2022/052905

§ 371 Date: May 15, 2024

(30) Foreign Application Priority Data
Nov. 16, 2021 (GB) 2116514.7

(51) Int. Cl. C12N 15/10; G01N 33/68; G16B 15/00; G16B 40/20

(52) U.S. Cl. CPC C12N 15/1089; G01N 33/6845; G16B 15/00; G16B 40/20

(57) ABSTRACT

A method for designing a chimeric antigen receptor (CAR), comprising: a) defining a training set of CAR sequences wherein each training CAR is associated with one or more properties; b) defining one or more objectives, each of the one or more objectives defining a desired property of a CAR; c) training a computational model using the training set of the one or more training CAR sequences to provide a trained computational model; d) using the computational model to provide at least one output CAR sequence, and wherein the at least one output CAR sequence is determined based on the one or more objectives. Methods of training a computational model for designing a chimeric antigen receptor (CAR), the trained model provided by such method, along with a CAR sequence output, the CAR encoded by the output CAR sequence and a cell expressing said CAR are also provided.

### FIELD OF THE INVENTION

The invention is in the field of chimeric antigen receptors (CARs), and in particular methods for the design and selection of CARs based on anticipated or predicted functionality.

### BACKGROUND OF THE INVENTION

Chimeric antigen receptors (CARs) are synthetic, engineered, membrane-bound receptors that are typically used to target surface molecules on other cells. CARs generally comprise an extra-cellular portion having a single chain variable fragment (scFv) that engages a target, a trans-membrane domain and an intracellular domain that is responsible for downstream signalling.

CARs have found use in the treatment of disease, particularly oncology, when present on a T-cell. Two CAR-T therapies, both directed to the B cell antigen CD19 have recently been approved by the FDA for use in the US.

To date, CARs are typically developed by first identifying and optimising an antibody to the target. This involves a variety of means, including preliminary antibody screening or panning, followed by characterization of any hits including sequencing the key recognition sequences. Once suitable antibodies are identified, typically based on affinity and/or specificity for the target alone, they may undergo additional levels of antibody optimization, such as through affinity maturation. At this point a small number will then be selected for further development in which the antigen binding regions (e.g. CDRs) are incorporated into a CAR and tested for functionality such as biological activity, toxicity, and cytokine production. If suitable functionality in the CAR is not achieved, the process must be repeated.

An inherent limitation in this process is that the selection of lead-clinical candidates is based mainly on criteria that select for and optimise good monoclonal antibodies, but not for the desired clinical end product, which is the CAR.

For a given target, the number of selected antibodies taken forward to incorporation in a CAR allowed for current cloning and testing Is around a maximum of ten CARs but more often only between two and five at a time. This number is low mainly because it is a resource intensive and labour-intensive process involving deconvoluting and sequencing the antibody, identifying the key sequences for recognition (CDRs), incorporating these into a suitable CAR scaffold and then manufacturing of viral particles, transduction of cells and assessing their functionality in a manual, low throughput manner. With a low number of CARs progressed, the chance of success of any one of those candidates is low meaning the process may need to be repeated, sometimes more than once. Each iteration of this process takes many months and, typically around 1-2 years.

The prior art shows that most researchers found 100+scFvs that target the antigen of interest but were only able to advance around 2-5 of these to a screen when incorporated into a CAR.

Examples of the standard CAR production processes are summarised in:
“Preclinical Evaluation of Allogeneic CAR T Cells Targeting BCMA for the Treatment of Multiple Myeloma”, Molecular Therapy, 2019.
US 2019/0161553 A1
US20170283504 A1

It is evident that this protracted approach also has a disproportionately high risk of failure as a functional antibody does not necessarily align with CAR functionality, such as for T-cell activation or cell killing when the CAR is expressed in an engineered immune cell, such as a T-cell. Highly functional antibodies can be poor performers when incorporated into a CAR due to many reasons, for example tonic signalling of the CAR (which is scFv dependent), epitope accessibility to CAR vs. mAb and biochemical stability of the fusion receptor. Ghorashian et al. (Nature Medicine, 2019) showed that lowering CAR affinity can result in increased serial killing and improved therapeutic performance. Contrary, other reports (e.g. Hudecek et al. Clinical Cancer Research, 2013) demonstrated that CARs based on high affinity scFvs showed greater anti-tumor potency compared to CARs with lower affinity scFvs. Thus, increased performance based on increased affinity of the scFvs for a target is not universal and depends on a multitude of interconnected factors such as antigen densities on target cells, CAR expression levels, and binding epitope location. None of these can be predicted based on antibody characteristics alone.

### Other Relevant Literature:

“CAR-T design: Elements and their synergistic function”, EBioMedicine, VOLUME 58, 102931, August 2020. (review of the challenges in CAR engineering and optimization)
Ghorashian, S., Kramer, A. M., Onuoha, S., Wright, G., Bartram, J., Richardson, R., et al (2019). Enhanced CAR T cell expansion and prolonged persistence in pediatric patients with ALL treated with a low-affinity CD19 CAR. Nature Medicine. https://doi.org/10.1038/s41591-019-0549-5 (lowering CAR affinity increases serial killing and therapeutic performance)
Liu, X., Jiang, S., Fang, C., Yang, S., Olalere, D., Pequignot, E. C., et al. (2015). Affinity-tuned ErbB2 or EGFR chimeric antigen receptor T cells exhibit an increased therapeutic index against tumors in mice. Cancer Research. https://doi.org/10.1158/0008-5472.CAN-15-0159. (lowering CAR affinity increases therapeutic index against tumors)
Hudecek, M., Lupo-Stanghellini, M. T., Kosasih, P. L., Sommermeyer, D., Jensen, M. C., Rader, C., & Riddell, S. R. (2013). Receptor affinity and extracellular domain modifications affect tumor recognition by ROR1-specific chimeric antigen receptor T cells. Clinical Cancer Research. https://doi.org/10.1158/1078-0432.CCR-13-0330 (high affinity CARs have increased anti-tumor efficacy)
Zhang, Z., Jiang, D., Yang, H., He, Z., Liu, X., Qin, W., et al. (2019). Modified CAR T cells targeting membrane-proximal epitope of mesothelin enhances the antitumor function against large solid tumor. Cell Death and Disease. https://doi.org/10.1038/s41419-019-1711-1. (Structural as well as functional aspects of the target epitope affect CAR functionality and should be included in design considerations for CARs).
James, S. E., Greenberg, P. D., Jensen, M. C., Lin, Y., Wang, J., Till, B. G., et al, Press, O. W. (2008). Antigen Sensitivity of CD22-Specific Chimeric TCR Is Modulated by Target Epitope Distance from the Cell Membrane. The Journal of Immunology. https://doi.org/10.4049/jimmunol.180.10.7028. (A membrane-distal epitope of CD22 was found to have weaker signaling, lower lytic efficiency, and defective degranulation compared to CARs binding to a membrane-proximal epitope)
Di Roberto, R. B. et al, “A functional Screening strategy for Engineering Chimeric Antigen Receptors with Reduced On-Target, Off-Tumor Activation; Molecular Therapy: Vol. 28(12), December 20 pp 2564-2576 describes the production of a ‘library’ of CARs and their screening. However, the method of preparing the library is limited by the process of preparing cells with “protoCARs”, i.e. CARs that do not have a full recognition site, instead having a partial recognition site and a locus for gene editing to incorporate a single point of variability in the recognition sequence (scFv). The paper relates solely to the optimisation of a known recognition sequence for certain benefits and fails to disclose a production of a CAR library or high throughput screening of a CAR library with multiple points of diversity in the recognition sequence and/or CAR scaffold.
US20190065677A1 and Briefings in Bioinformatics, Volume 21, Issue 5, September 2020, Pages 1549-1567, each describe use of machine learning techniques to identify antibodies. This is a relatively simple two component interaction model, and does not have the same complexity of a CAR which comprises and antigen binding moiety, a hinge region and an intra-cellular signalling region.

Other Machine learning approaches to the design of CAR-Ts, such as Daniels et al. are described in International application no. WO2022173703 and bioRxiv preprint doi: https://doi.org/10.1101/2022.01.04.474985, which was published after the priority date of the present application.

Exploring the rules of chimeric antigen receptor phenotypic output using combinatorial signaling motif libraries and machine learning), have, to date, only focused on the embedding of intracellular domains and predicting novel combinations, whereas these methods would fail to successfully predict how these would affect the overall function and stability, avidity and efficacy of the CAR.

Therefore, a need remains to identify methods and systems which the function of a CAR can be evaluated based on an information-rich, targeted design approach. Such an approach may employ computational and/or Artificial Intelligence (AI) and/or machine learning approaches to develop and/or iterate the design process in-silico to predict and desirable CAR functionality.

### SUMMARY OF THE INVENTION

In a first aspect, the invention provides a method for designing a chimeric antigen receptor (CAR), comprising:
a) defining a training set of one or more training CAR sequences, each of the one or more training CAR sequences in the training set encoding a training CAR, wherein each training CAR is associated with one or more properties:
b) defining one or more objectives, each of the one or more objectives defining a desired property of a CAR;
c) training a computational model using the training set of the one or more training CAR sequences to provide a trained computational model that outputs an approximation of the one or more properties of a CAR as a function of one or more features of a CAR sequence;
d) using the computational model to provide at least one output CAR sequence, wherein the at least one output CAR sequence is not in the training set, and wherein the at least one output CAR sequence is determined based on the one or more objectives, and wherein the at least one output CAR sequence encodes a CAR.

In a second aspect, the invention provides a method for training a computational model for designing a chimeric antigen receptor (CAR), comprising:
a) defining a training set of one or more training CAR sequences, each of the one or more training CAR sequences in the training set encoding a training CAR, wherein each training CAR is associated with one or more properties:
b) defining one or more objectives, each of the one or more objectives defining a desired property of a CAR;
c) training a computational model using the training set of the one or more training CAR sequences to provide a trained computational model that outputs an approximation of the one or more properties of a CAR as a function of one or more features of a CAR sequence.

In a third aspect, the invention provides a trained computational model prepared by the method of any one of the first or the second aspect.

In a fourth aspect, the invention provides a CAR sequence output by the method of the first aspect.

In a fifth aspect, the invention provides a CAR encoded by the CAR sequence of the fourth aspect.

In a sixth aspect, the invention provides a cell expressing the CAR of the fifth aspect.

In a seventh aspect, the invention provides a non-transitory, computer-readable storage medium storing instructions thereon that when executed by a computer processor causes the computer processor to perform the method of the first or second aspect.

In an eighth aspect, the invention provides a computing device, comprising:
an input arranged to receive:
i) data indicative of a training set of training CAR sequences, each training CAR sequence comprising an antigen binding domain; a hinge domain; a transmembrane domain; and an intracellular domain, wherein each training CAR sequence is associated with one or more properties; and
ii) data indicative of one or more objectives each of the one or more objectives defining a desired property;
a processor arranged to train, using the training set of training CARs, a computational model to provide an approximation of properties of a CAR as an function of one or more features of the CAR, and arranged to output at least one output CAR, which are not in the training set, and wherein the at least one output CAR sequence is determined based on the one or more objectives, and wherein the at least one output CAR sequence encodes a CAR; and
an output arranged to output the determined subset.

In a ninth aspect, the invention provides at least one non-transitory computer-readable storage medium storing computer-executable instructions that, when executed by at least one processor, cause the at least one processor to perform a method for identifying an amino acid sequence for a chimeric antigen receptor (CAR), wherein said amnio acid sequence comprises an antigen binding domain sequences; a hinge domain sequence; a transmembrane domain sequence; and an intracellular domain sequence, the method comprising:
querying a machine learning engine for a proposed amino acid sequence for a chimeric antigen receptor (CAR) having a high desired functionality, wherein the machine learning engine was trained using functionality information for different amino acid sequences for a chimeric antigen receptor (CAR); and
receiving from the machine learning engine the proposed amino acid sequence for a chimeric antigen receptor (CAR), the proposed amino acid sequence indicating a specific amino acid for each residue of the proposed amino acid sequence.

In a tenth aspect, the invention provides a computer-implemented method for identifying an amino acid sequence for a chimeric antigen receptor (CAR), wherein said amnio acid sequence comprises one or more antigen binding domain sequences; a hinge domain sequence; a transmembrane domain sequence; and an intracellular domain sequence, the method comprising:
querying a machine learning engine for a proposed amino acid sequence for a chimeric antigen receptor (CAR) having a desired functionality, wherein the machine learning engine was trained using functionality information for different amino acid sequences for a chimeric antigen receptor (CAR); and
receiving from the machine learning engine the proposed amino acid sequence for a chimeric antigen receptor (CAR), the proposed amino acid sequence indicating a specific amino acid for each residue of the proposed amino acid sequence.

In an eleventh aspect, the invention provides a system comprising control circuitry configured to perform a computer implemented method for identifying an amino acid sequence for a chimeric antigen receptor (CAR), wherein said amnio acid sequence comprises an antigen binding domain sequence; a hinge domain sequence; a transmembrane domain sequence; and an intracellular domain sequence, the method comprising:
receiving an initial amino acid sequence for a CAR having at least one characteristic;
training a machine learning engine using data that includes a plurality of amino acid sequences for a chimeric antigen receptor (CAR) and information identifying the at least one characteristic corresponding to each of the plurality of amino acid sequences; and
querying the trained machine learning engine for a proposed amino acid sequence for a chimeric antigen receptor (CAR) having functionality at the target, wherein the proposed amino acid sequence differs from the initial amino acid sequence, wherein the querying the machine learning engine comprises:
predicting the proposed amino acid sequence by using the at least one characteristic to identify a specific amino acid for at least one residue of the proposed amino acid sequence; and
receiving from the machine learning engine the proposed amino acid sequence.

In a twelfth aspect, the invention relates to a computer-implemented method for the design of CARs, the method comprising:
Generating a modular sequence space of one or more of:
   binding moieties (e.g. scFv, VHH, receptor-based)
   hinge regions (domains, sequences)
   transmembrane regions (domains, sequences)
   intracellular regions (domains, sequences) (e.g. co-stimulatory, stimulatory, inhibitory, activatory, positive feedback loops, negative feedback loops); and optionally
   linker regions (domains, sequences) for connecting one or more of the above regions, suitably for connecting the hinge region to the transmembrane region.
Generating a CAR sequence space comprising a plurality of CAR sequences generated from combinations of one or more of the above modular sequence spaces.
Selection of a subset of CAR sequences from the CAR sequence space based on anticipated functionality.

In embodiments, the method of the tenth aspect wherein when less than all of the modular sequence spaces are used, the remaining sequence(s) for any given region (domain, sequence) of the CAR sequence or CAR sequence space may be obtained from any other suitable source (proprietary dataset, commercial dataset, individual sequence etc.).

In embodiments, the anticipated functionality may be based on suitable machine learning methods or other computational tools. In embodiments, such machine learning tools filter for trained observations and eliminate CARs with poor expected functionality, such as protein stability, tonic activation, aggregation propensity, non-accessible epitopes, non-optimal epitopes, low avidity, low specificity, low activation propensity, non-optimal T cell activation.

In a thirteeth aspect, the invention relates to a computer-implemented method for generating in-silico CAR sequence space by varying multiple components of the CAR sequence individually or in combination, the method comprising:
Generating a modular sequence space of one or more of:
   binding moieties (e.g. scFv, VHH, receptor-based)
   hinge regions (domains, sequences)
   transmembrane regions (domains, sequences)
   intracellular regions (domains, sequences) (e.g. co-stimulatory, stimulatory, inhibitory, activatory, positive feedback loops, negative feedback loops); and optionally
   linker regions (domains, sequences) for connecting one or more of the above regions, suitably for connecting the hinge region to the transmembrane region.
Generating a CAR sequence space comprising a plurality of CAR sequences generated from combinations of one or more of the above modular sequence spaces of a CAR.

In a fourteenth aspect, the invention provides a CAR sequence selected by the method of the tenth aspect or the eleventh aspect.

In a fifteenth aspect, the invention provides a CAR encoded by the CAR sequence of the twelfth aspect.

In a sixteenth aspect, the invention provides a cell expressing the CAR of the thirteenth aspect.

In a seventeenth aspect, the invention provides a non-transitory, computer-readable storage medium storing instructions thereon that when executed by a computer processor causes the computer processor to perform the method of the tenth aspect or the eleventh aspect of the invention.

### BRIEF DESCRIPTION OF THE FIGURES

FIG. 1: Schematic showing the process steps of an embodiment of the present invention to allow prediction of novel CAR sequences with desired properties.

FIG. 2: Predicted novel CAR sequences can be synthesized or assembled. CAR sequences may then be tested to assess functional outputs, such as CAR expression, target binding, affinity, avidity, cytokine production, cytotoxicity, tonic activation, proliferation and others. The data may be incorporated into a database, associating the tested CAR sequences with the functional output values. This may be used to train computational models and design new CAR sequences. This cycle can be repeated multiple times in order to improve predictions further for a given output.

FIG. 3: CAR sequences were embedded using Sapiens natural language models and visualised. Axes represent UMAP two dimensional coordinates. Shading indicates target and dot size indicates CAR-T activation score.

FIG. 4: CAR sequences were designed against BCMA in accordance with an embodiment of the invention (black). An anti-BCMA CAR sequence was used as input to predict sensible alternative sequences of this CAR. These sequences were benchmarked against an initial, proprietary best-in-class anti-BCMA CAR (highlighted by arrow). The CAR sequences were transduced into T cells and after a 6-day expansion, they were assessed for affinity/avidity of the CAR. CAR-T cells were incubated with a low concentration of BCMA-Fc protein and subsequently stained with anti-Fc-PE antibody. The median fluorescence intensity (indicative of binding intensity) of anti-Fc-PE was measured. Dashed line shows the median fluorescence intensity of the lead anti-BCMA candidate.

FIG. 5: CAR-T activation. Designed CAR-T cells (black), control anti-CD19 CAR T-cells (highlighted by dashed arrow) and lead candidate anti-BCMA CAR T-cells (highlighted by arrow) were tested for their potency against the BCMA-expressing RPMI-8226 cell line. After incubation for 24h, cells were stained for different activation markers, such as CD69, CD25 or cytokines (e.g. IFNg). Here, % CD69+ cells are shown. Dashed line shows the % CD69+ of the lead anti-BCMA candidate.

### DETAILED DESCRIPTION OF THE INVENTION

All references cited herein are incorporated by reference in their entirety. Unless otherwise defined, all technical and scientific terms used herein have the same meaning as commonly understood by one of ordinary skill in the art to which this invention belongs.

Prior to further setting forth the invention, a number of definitions are provided that will assist in the understanding of the invention.

The articles “a” “an” and “the” are used to refer to one or to more than one (i.e. to at least one) of the grammatical object of the article.

As used herein, the term “comprising” means any of the recited elements are necessarily included and other elements may optionally be included as well. “Consisting essentially of” means any recited elements are necessarily included, elements which would materially affect the basic and novel characteristics of the listed elements are excluded, and other elements may optionally be included. “Consisting of” means that all elements other than those listed are excluded. Embodiments defined by each of these terms are within the scope of this invention.

Unless specifically defined herein, all terms used herein have the same meaning as they would to one skilled in the art of the present invention. Practitioners are particularly directed to Green and Sambrook, Molecular Cloning: A Laboratory Manual, 4th ed., Cold Spring Harbor Laboratory Press, New York (2012); and Ausubel et al., Current Protocols in Molecular Biology (up to Supplement 114), John Wiley & Sons, New York (2016), for definitions and terms of the art. The definitions provided herein should not be construed to have a scope less than understood by a person of ordinary skill in the art.

A “chimeric antigen receptor” or “CAR” as used herein refers to a chimeric receptor (i.e. a receptor composed of two or more parts from different sources) that has at least a binding moiety or recognition sequence with a specificity for a target such as an antigen or protein, a hinge region, a transmembrane portion and an intracellular signaling domain that can invoke a signal in the cell in which the CAR is present (e.g. a CD3 zeta chain).

As used herein the term ‘antigen binding domain’ refers to a peptide sequence that is intended or able to bind a target of interest. In examples, the antigen binding domain is an antigen-binding fragment as defined above. All types of antigen binding domains are encompassed by the present invention. Examples of some antigen binding domains are scFvs, VHH single domain antibodies or nanobodies, and antigen binding fragments.

An ‘scFv’ or ‘single chain variable fragment’ as used herein, refers to a type of antigen binding domain. Typically, an scFv is a fusion of the variable regions of the heavy (VH) and light chains (VL) of an antibody for a given target connected by a short linker. The VH and VL regions may be in any order around the linker, for example, the scFv may have (i) a first VH chain, a linker and a VL chain or (ii) a first VL chain, a linker and a VH chain. As it is generally accepted that both versions would lead to similar activity, both are encompassed by the present disclosure even in the event that only one is exemplified. Antigen binding domains may comprise ‘CDRs' or’complementarity determining regions' which are predominantly responsible for target binding. On a typical antibody, multiple CDRs exist and may be selected or varied independently to achieve multiple points of diversity.

As used herein the term “recognition sequence” refers to the nucleic acid sequence encoding for a complementary peptide sequence that is intended or able to bind a target of interest. All types of recognition sequences are encompassed by the present invention.

Examples of some recognition sequences are scFvs VHH single domain antibodies or nanobodies, and antigen binding fragments. An “scFv” of “single chain variable fragment” is a type of recognition sequence. Typically, an scFv is a fusion of the variable regions of the heavy (V_{H}) and light chains (V_{L}) of an antibody for a given target connected by a short linker. Recognition sites may comprise “CDRs” or “complimentary determining regions” which are predominantly responsible for target binding. On a typical antibody, multiple CDRs exist and may be selected or varied independently to achieve multiple points of diversity.

A “transmembrane domain” or “TM domain” as used herein is any membrane-spanning protein domain. Suitably, the TM domain in a CAR is derived from a known transmembrane protein sequence. However, it can also be artificially designed.

As used herein the term “hinge domain” refers to a peptide sequence that connects the antigen binding domain and transmembrane region of a CAR. The hinge domain is located between the antigen binding fragment and the T cell plasma membrane (Moritz D, et al. Gene Ther. 1995; 2(8):539-46).

The term “signaling domain” or “intracellular domain” or “intracellular signaling domain” as used herein refers to a moiety that can transmit a signal in a cell, for example an immune cell. The signaling domain typically comprises a domain derived from a receptor that signals by itself in immune cells, such as the T Cell Receptor (TCR) complex or the Fc receptor or DAP10/DAP12 receptors. Additionally, it may contain a costimulatory domain (i.e. a domain derived from a receptor that is required in addition to the TCR to obtain full activation, or the full spectrum of the signal in case of inhibitory costimulatory domains, of T cells). The costimulatory domain can be from an activating costimulatory receptor or from an inhibitory costimulatory receptor.

“Antibody” refers to all isotypes of immunoglobulins (IgG, IgA, IgE, IgM, IgD, and IgY) including various monomeric, polymeric and chimeric forms, unless otherwise specified.

Specifically encompassed by the term “antibody” are polyclonal antibodies, monoclonal antibodies (mAbs), single domain antibodies, human (FHVH) or heavy-chain antibodies found in camelids (VHH) and antibody-like polypeptides, such as chimeric antibodies and humanized antibodies. “Antigen-binding fragments” are any proteinaceous structure that may exhibit binding affinity for a particular antigen. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)2, Fc, Fabc, and Fv molecules, single chain (sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CHI domains, or a monovalent antibody as described in WO2007059782, bivalent fragments comprising two Fab fragments linked by a disulfide bridge at the hinge region, a Fd fragment consisting essentially of the V.sub.H and C.sub.HI domains; a Fv fragment consisting essentially of the VL and VH domains of a single arm of an antibody, a dAb fragment (Ward et al., Nature 341, 544-546 (1989)), which consists essentially of a VH domain and also called domain antibodies (Holt et al: Trends Biotechnol. 2003 November; 21(11):484-90); camelid or nanobodies (Revets et al; Expert Opin Biol Ther. 2005 January; 5(1): 111-24); an isolated complementarity determining region (CDR), and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “an antibody or antigen-binding fragment thereof may be used to denote that a given antigen-binding fragment incorporates one or more amino acid segments of the antibody referred to in the phrase.

“Specific binding” or “immunospecific binding” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a KD of less than about 1×10⁻⁸ M, as measured by a surface plasmon resonance assay or a cell binding assay. Phrases such as “[antigen]-specific” antibody (e.g., BCMA-specific antibody) are meant to convey that the recited antibody specifically binds the recited antigen.

As used herein, the terms “bi-specific”, “tri-specific” or “multi-specific” refer to an antibody molecule (i.e. an antibody or antigen binding fragment conjugated to a synthetic molecule) that comprises one or more further antigen binding domains such that the antibody molecule can have specificity for more than one antigen.

The phrase “nucleic acid molecule” synonymously referred to as “nucleotides” or “nucleic acids” or “polynucleotide” refers to any polyribonucleotide or polydeoxyribonucleotide, which may be unmodified RNA or DNA or modified RNA or DNA. Nucleic acid molecules include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

There are various means by which a nucleic acid sequence may be inserted into a genome, including but not limited to plasmid or vector transfection, transposition and genome editing. All are contemplated for use in the present invention. A “vector” is a replicon, such as plasmid, phage, cosmid, or virus in which another nucleic acid segment may be operably inserted so as to bring about the replication or expression of the segment. A “transposon” or “transposable elements” are DNA sequences that can change their position within a genome. “Genome editing” refers to the ability to edit the genome to insert the required sequence, for example using CRISPR-Cas9 genome editing technology.

As used herein, a “clone” is a population of cells derived from a single cell or common ancestor by mitosis.

A “cell line” is a clone of a primary cell that is capable of stable growth in vitro for many generations. In some examples provided herein, cells are transformed by transfecting the cells with DNA.

The terms “express” and “produce” are used synonymously herein and refer to the biosynthesis of a gene product. These terms encompass the transcription of a gene into RNA. These terms also encompass translation of RNA into one or more polypeptides, and further encompass all naturally occurring post-transcriptional and post-translational modifications.

A “point of diversity” of a CAR or CAR library or CAR-cell library as used herein means a component or region in the structure of a CAR that may be varied to modulate or optimise its function. A point of diversity may comprise one or more regions of the binding moiety or recognition sequence, and/or the choice or adaptation of one or more components of the CAR scaffold, such as the hinge region, a transmembrane portion and an intracellular domain.

The term “subject” refers to human and non-human animals, including all vertebrates, e.g., mammals and non-mammals, such as non-human primates, mice, rabbits, sheep, goats, dogs, cats, horses, cows, chickens, amphibians, and reptiles. In most particular embodiments of the described methods, the subject is a human.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters including the results of a physical examination, neurological examination, or psychiatric evaluations.

As used herein, the term ‘AI’ or ‘artificial intelligence’ means is the capability of a computer system to mimic human cognitive functions such as learning and problem-solving.

As used herein, the term ‘machine learning’ is an application of AI and means the use and development of computer systems that are able to learn and adapt without following explicit instructions, by using algorithms and statistical models to analyse and draw inferences from patterns in data.

As used herein, the term ‘deep learning’ means a type of machine learning based on artificial neural networks in which multiple layers of processing are used to extract progressively higher-level features from data.

As used herein, the term “inference” or “machine learning inference” means the ability of a system to make predictions from novel data.

As used herein, the term ‘embed’ or ‘embedding’ or ‘embedded’ means (the process, or the result of a process, of transforming a protein sequence, such as a CAR sequence or pail thereof, into a set of numbers—a numerical vector which generally has a lower dimensional representation.

As used herein, the term “properties” refers to one or more functional outcomes of a CAR. Suitably properties may refer to the: binding affinity of a CAR to a target; avidity, specificity; selectivity; cytotoxicity; cellular activation; intracellular signalling; tonic signalling, expression, surface expression, cytokine production, proliferation, exhaustion, serial killing ability of a cell in which the CAR is expressed; Kₒₙ/k_{off} rates of a cell in which the CAR is expressed; target binding location; epitope; and any combination thereof.

As used herein, the term “features” refers to one or more characteristics of fragments of a sequence that may be associated with a particular property. Features in this context may be, in the sequence or part thereof, in particular, the antibody binding domain (scFv, VHH) sequence, the hinge domain sequence, the linker sequence, the transmembrane domain sequence and the intracellular domain sequence (co-stimulatory, stimulatory, inhibitory, activatory, positive feedback loops, negative feedback loops) and their parts or fragments or combinations; 3D structure; primary structure of the CAR; secondary structure of the CAR; tertiary structure of the CAR; polarity; hydrophobicity; electrostatics; protein stability; thermal stability; vector distance between atoms or amino acids; linear distance between atoms or atoms; or any combination thereof.

The invention relates, in one aspect, to a method, suitably a computational or in-silico method, for the design and prediction of functionality of a CAR, and optionally use of this method to design or select or generate the best candidates for further in-silico or real-world evaluation.

To date, the development of a CAR, for example for CAR-T cell therapies, has followed the accepted process of (1) selecting an antigen target; (2) identifying an antibody for the given antigen target, typically this is by some form of antibody enrichment procedure such as screening or panning a phage display, immunisation or yeast display library, for example, and optionally further optimising the antibody to develop the antigen binding properties; (3) characterising the antibody specificity, affinity and to identify the sequence of the complimentary recognition sequences (CDRs); (4) incorporate the selected scFv or the CDR sequence into a scFv of a CAR, along with a choice of transmembrane domain and intracellular signalling domain; (5) evaluate the properties of the CAR in in-vitro and then a clinical context.

This process suffers from a number of significant drawbacks.

Firstly, the protracted process of isolating and characterising antibodies from the initial screen in step (2) is both labour and resource intensive. This limits the number of CARs that can be prepared and evaluated from the initial screen.

Secondly, isolating and characterising antibodies brings forward costs and effort to the front end of the process so that considerable screening effort is spent on understanding and characterising the antibody, despite the fact that this is not the desired clinical product.

Thirdly, antibody activity in vitro does not always translate to equivalent CAR activity in a clinical context. This can mean a given antibody with promising baseline activity is progressed for evaluation as a CAR where it can fail to show the desired properties in a clinical context. Indeed, this is one of the known areas of failure in conventional CAR development processes which priorities antigen binding affinity at the early stage over clinical efficacy of the ultimate CAR T cell product.

Fourthly, the ability to vary multiple potential points of diversity in the CAR is severely limited by the number of CARs produced, and the stepwise process in which CAR development is conducted. The recognition domain, including each individual CDR in the CAR recognition sequence, and/or other parts of the CAR scaffold (hinge region, transmembrane domain and intracellular domain) may have an impact on CAR function that is difficult to predict, and must be tested.

Indeed, the present invention is based, at least in part. upon the understanding that antibody characteristics, such as affinity, are not the only factors driving functionality of CARs. Other factors are important in the developability of a CAR, such as:
protein stability;
immunogenicity;
expression of the CAR on the cell surface of relevant cells (compared to e.g. phage display or yeast display);
avidity;
aggregation potential;
tonic signaling of the CAR, which can be caused by aggregation of the receptor/ScFv, access to the epitope;
distance between the CAR-cell and the target epitope;
the target protein/epitope/target expression level.

The present inventors have appreciated that the accepted process for the identification of a CAR clinical candidate may be rationalised so that resources are focussed primarily on synthesis and characterisation of the best clinical candidate as early as possible in the development process.

One approach to the above problem is to have the ability to physically screen a large library of CAR-expressing cells with high diversity, in a similar, but distinct, manner to antibody libraries (˜10⁸ to ˜10¹³). This is the subject of the Applicant's patent application no. PCT/GB2022/050158. While this approach is sound and offers many benefits over the current state of the art, it still requires the physical preparation of a large number of potential CAR candidates, generated from the combination of potential domains and optionally expression in cells, which, in some applications, can still be time and labour intensive, albeit a dramatic improvement over the current state of the art.

An alternative approach that forms the basis of the present disclosure is the use of computational, AI and/or machine learning tools to specifically design CARs and/or parts thereof, such as antigen binding domains, or intracellular domains that are precited to have desired or beneficial properties. These tools can help discard candidates with poor developability potential, and/or focus on and select those with the desired developability characteristics, such as:
epitope location
tonic signalling
protein stability
protein expression
affinity
avidity
optimal hinge length
optimal intracellular signalling domain
optimal transmembrane domain

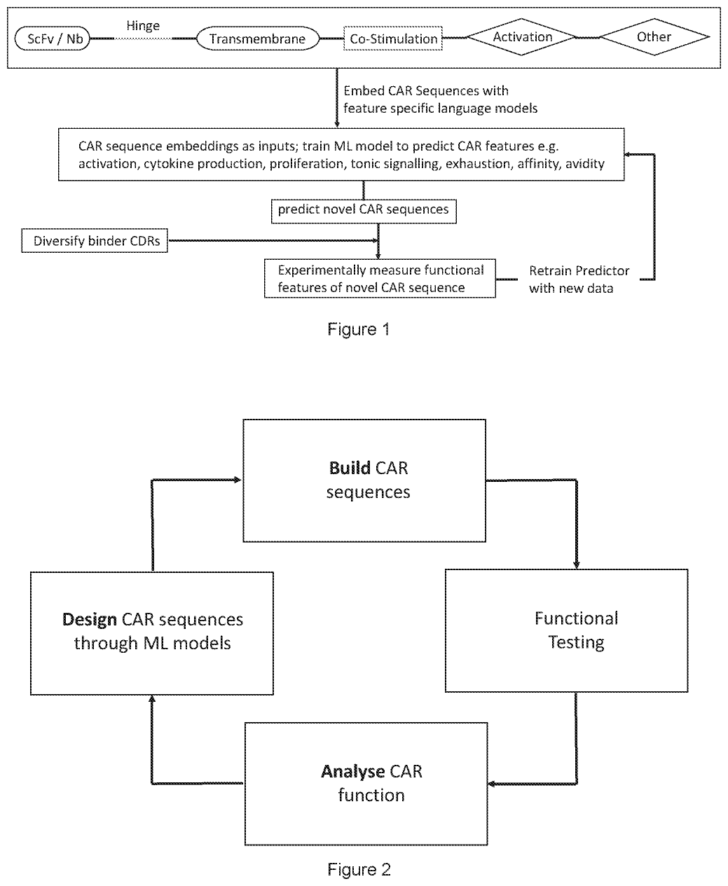

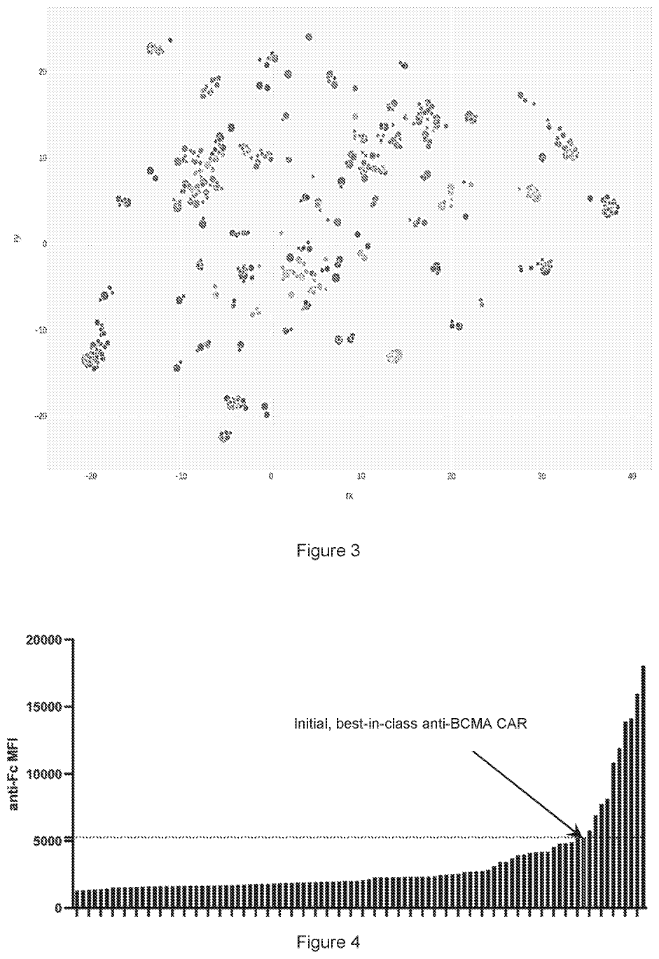

The general concept relies on the use of deep learning tools using and/or combining information-rich training datasets to efficiently identify CAR sequences of high clinical potential allowing the selection of a limited number of potential CARs to be tested, those CARs having an increased probability of the desired functionality. Built into the method is the ability to iterate the design process until a CAR of the desired functional properties is obtained. An embodiment of the general process is shown in FIG. 1 and FIG. 2, and a discussion of how such machine learning techniques may be applied to in-silico CAR design is provided below.

Generating training datasets of CAR sequences associated with functional data

An important aspect of the present invention is the ability to provide the desired computational models with a large training set of CAR sequences associated with properties and/or measurable/observable/predicted data based on evaluation of the CARs they encode in wet, real-world experiments.

CAR sequences can be tested to assess functional outputs, such as CAR expression, target binding, affinity, avidity, cytokine production, cytotoxicity, tonic activation, proliferation and others. The data may be incorporated by any suitable means, for example, into a database, which then associates the tested CAR sequences with functional output values. This may be used to train computational models and design new CAR sequences. This cycle can be repeated multiple times in order to improve predictions further for a given output.

Any suitable method of providing a set of CAR sequences associated with functional outcomes or measurable or observable data is envisaged.

In an embodiment of the present invention, in a first step a wide range of CAR sequences are expressed in a suitable cell line. Suitably, the CAR sequences are transfected, transduced or electroporated using standard methods into a suitable population of cells or a cell line. The cells generate are functionally characterised by any suitable means, for example, using single cell (SC) data, population sorting by flow cytometer or magnetic enrichment, biological enrichment (e.g. persistency, proliferation) and/or imaging methods. One suitable example of such a library preparation may be found in the Applicant's patent application no. PCT/GB2022/050158, the entire contents of which is incorporated herein by reference, or specifically, the method of preparing a library and collecting data.

In embodiments, the number of CARs expressed and evaluated may be a minimum of 100, 500, 1000, 5000, 10000, 50000, 100000, 500,000, 1,000,000, 10,000,000 or more. There is no limitation on upper limit of the number of CARs and their associated sequences that may be used in the training dataset, and this is limited only by the ability to generate the CARs and functional data.

In embodiments, the dataset collected includes the sequence of the CAR linked to functional outcomes, such as, but not necessarily limited to, levels of: CAR activation; cytokine production; degranulation; cell proliferation; activation of signalling pathways downstream of the CAR; tonic signalling; effector memory phenotype; CAR expression level; CAR affinity; CAR avidity; exhaustion signature; and/or activation signature.

In specific embodiments, a number of options for training data are envisaged:

Embodiment 1: the training dataset is based on CAR sequence information and information about functional outcomes (as defined immediately above).

Embodiment 2: the training dataset is based CAR sequence information, knowing the target of the CAR and functional outcomes (as defined immediately above).

In embodiments, CARs can be tested on a range of different methodologies, pooled or in parallel (as described in the Applicant's patent application no. PCT/GB2022/050158).

In embodiments, the training dataset generated links CAR sequences to functional activity. This includes single, dual, triple- or other multi-specific CARS as, in embodiments, the approach can be applied to multi-specific binders.

In embodiments, the dataset generated against the functionally annotated CAR sequences is then used to train suitable computational models and/or machine learning algorithms. In embodiments, these models may be trained to predict CAR functional outcomes from the CAR sequence.

### Training the Computational Model

Any suitable method may be used for the computational model. Suitable models may be, for example, language models or denoising diffusion models.

In one embodiment, machine learning inference models may be used to select from a provided set of CAR sequences. Alternatively, or in addition, similar suitably trained machine leaning inference models may be used to select optimal or improved combinations of CAR domains from sets of individual or linked CAR domains, selected from antigen binding domains; hinge domains; transmembrane domains; intracellular domains and optionally co-stimulatory domains, or combinations thereof.

In a further embodiment, suitably trained deep learning models may be used to generate CAR sequences with desired predicted properties. The generation of CAR sequences by these models may be de novo (i.e. generating a CAR sequence without any user provided start point), or a start or seed CAR sequence (or part thereof, may be used as a start point for generating a novel, optimised or improved CAR sequence.

In a specific embodiment, in order to predict CAR functional outcome from the CAR sequence, deep learning methods may be used to embed the CAR sequence into vector representations using the state of the art (SOTA) protein embedding method (e.g. currently Prot-T5).

In any of the above embodiments, the computational model may be pre-trained with a relevant dataset that may improve the desired output. This pre-training may be more general or rough, for example, training the model to recognise protein structure, CAR structures, antibody structures etc. Use of the CAR sequence dataset associated with one or more properties may then be used to fine tune the model.

In embodiments, the training of the model may be sequential with one or more further datasets or input data, or it may be simultaneous. Suitably, the pre-training is conducted ahead of fine tuning with the CAR sequence data associated with functional data. Certain models pre-trained may be available commercially or open source, such as transformer based language models Prot-T5, prot-BERT, MCSM, TAPE or structural based ones such as DiffAb, Ab-dock-gen, FV hallucinator or antibody based ones, such as SapienS, ABlang.

In a specific embodiment, the antibody region (or recognition region or scFv region or VHH region) may be embedded using a model pre-trained on antibody sequences and their complementarity-determining region (CDR) sequences such as the ones naturally occurring in the human antibody repertoire or in other animals such as Alpaca or Llama for VHH domains. In other embodiments binding domains may be embedded using a model pre-trained on receptor-ligand interactions. In other embodiments, any region, domain or part of the CAR may be embedded using a model further pre-trained on known, or predicted, existing corresponding domains.

In embodiments, the residue (e.g. amino acid) level embeddings are reduced to a lower dimensional representation using a suitable technique, such as an autoencoder. In embodiments, the resulting sequence representations may be used to train a suitable computational method or device to predict CAR functionality from the sequence representation, and by extension, to the original sequence.

In embodiments, the trained model may then be used to select, build or generate, in-silico or computationally, potential improvements in relevant CAR sequence features including, but not limited to, in the antibody (scFv, VHH) domain, hinge domain, linker, transmembrane domain and intracellular domain (co-stimulatory, stimulatory, inhibitory, activatory, positive feedback loops, negative feedback loops) and their combinations.

Such improvements in features, based on a predictive improvement in one or more desired properties of the CAR, may be used alone or combined to prioritise CAR sequences for preparations and testing in the lab.

An example of such an improvement may be in the antigen binding domain. Other improvements may be in the combination of a selection of domains, or other fragments of the CAR sequence.

A further example of such an improvement may be in the intracellular domain.

A CAR activates a variety of the T cells intracellular downstream signalling pathways. The activation of the downstream pathways can be designed by the selection and specification of intracellular signalling domains of the CAR.

Currently, CD247 stimulatory domains and 4-1BB and/or CD28 are used and activate a broad range of intracellular signalling events (e.g. NFAT activation, Nf-kB signalling, ERK signalling), resulting in T cell activation, proliferation, cytokine production, cytotoxicity and/or cell differentiation at the same time.

In embodiments of the present invention, synthetic intracellular domains can be predicted resulting in e.g. only cell survival and/or only cell proliferation and/or only cytotoxicity. Alternatively, intracellular domains can prevent T cell activation through negative signals (e.g. ITIM).

Specific embodiments of training the model as envisaged for the present invention are as follows:

Embodiment 1: Pre-train the model (e.g. language model) on protein sequences (naturally occurring or synthetic proteins, antibody (VH/VL1scFv/VHH) and in house CARs) before fine tuning the model on CAR sequence functional data (for example a regression model or a classifier). Protein language models “understand” how a “functional” protein should look like and how to embed these in the model.

Embodiment 2: Pre-train the model on antibody sequence before fine tuning on CAR sequence functional data (for example, a regression model or a classifier). Pre-training in this way assists the model in “understanding” the syntax of what an antibody, in general, should look like.

Embodiment 3: Directly train on CAR sequences and functional data; (for example, a regression model or a classifier).

Embodiment 4: Train the model on CAR sequences, linked to function and optionally target information (e.g. target sequence or target structural information. Such models may employ neural networks such as ENN, (equivariant neural networks), GNN (graph neural networks).

Training of the model enabled enables it to approximate function of a CAR to one or more features, suitable structural features of a CAR.

Suitably, the features of a CAR and/or CAR sequence are selected from the group consisting of: sequence; part of the sequence; primary structure of the CAR; secondary structure of the CAR; tertiary structure of the CAR; polarity; hydrophobicity; electrostatics; vector distance between atoms or amino acids; linear distance between atoms or atoms; or any combination thereof.

### Generating Novel CAR Sequences

Once suitably trained, the model can be used to generate one or more output CAR sequences, suitably, these output CAR sequences are not in the training set, however, on occasion, the model may output a CAR sequence that meets the objectives that is already in the training set.

In embodiments, the trained model outputs CAR sequences that are predictive of attainment of, or improvements in, one or more defined functions of a CAR. Such ranking may be defined by setting one or more objectives for the model, each objective relating to a desired function of a CAR. The model then, based on the training, can then output CAR sequences that are predictive of best meeting these objectives.

In embodiments, the model may output CAR sequences based on varying inputs. In one example, the model may be provided with a screening set comprising CAR sequences, these CAR sequences not intended to be present within the training set. Based on the defined objectives, the model may then select a subset of CAR sequences from the screening set that are predicted to best achieve the desired functional outcome.

In a related embodiment, a similar selection may be made from sets of domains or other fragments of a CAR sequence, such as the antigen binding domain, the hinge domain, the linker domain, the intracellular domain and/or the co-stimulatory domain, or any combination thereof that is suitable for recombination in a CAR sequence (i.e. the domains are linearly combined in a manner as present in a native CAR, such as the antigen binding domain being joined to the hinge domain, and not directly to the intracellular domain).

In a further embodiment, the model may generate a CAR sequence for output that has been designed de novo (i.e. with not input provided by the operator), or by optimisation from a start or seed CAR sequence, wherein the start or seed CAR sequences is a full CAR sequence comprising an antigen binding domain, a hinge domain, a transmembrane domain an intracellular domain and optionally a co-stimulatory domain, or part thereof.

Specific embodiments of the generation of CAR sequences may be:
Embodiment 1: Generate full CAR sequences de novo wherein, the full CAR sequence comprises an antigen binding domain, a hinge domain, a linker domain, an intracellular domain and optionally a co-stimulatory domain;
Embodiment 2: input a starting CAR sequence and optimise the full sequence;
Embodiment 3: input a part of a CAR sequence, such as an antigen (target) sequence and generate CARs against that sequence part;
Embodiment 4: input a starting CAR sequence and optimise defined regions of the CAR (e.g. hinge, binding etc.);
Embodiment 5: input one or more domain sequences of a CAR (e.g. binder) and generate the remainder of the CAR.

In each of the above embodiments, the process can be run in an iterative fashion where output CARs from one run of the model can be fed back into the training set, further refining the model, before running it a second, or more, times in a similar manner.

### In Silico CAR Optimization

### REFERENCES

Antibody design using LSTM based deep generative model from phage display library for affinity maturation; Scientific Reports volume 11, Article number: 5852 (2021)
A Review of Deep Learning Methods for Antibodies; PMID: 32354020
In Silico Proof of Principle of Machine Learning-Based Antibody Design at Unconstrained Scale; bioRxiv, doi:10.1101/2021.07.08.451480, 2021-07-09.

In further embodiments, the set of in-silico CARs generated by the method described above may be expanded by diversifying certain regions, for example the CDR regions in the antigen binding domain using an additional computational, AI and/or machine learning methods.

In embodiments, this additional computational, AI and/or machine learning method is a model, suitably a proprietary language model, pre-trained on a dataset, suitably a large dataset of more than a million, suitably many millions of relevant sequences, such as CDR sequences to allow for improved understanding of structure and function for a given region of the CAR, such as CDR structure and function.

In embodiments, such a model may then be jointly trained with a smaller dataset of thousands of sequences known to have the desired function, for example CDRs with known antigen partners, in a structurally aware manner by combining a language model and a neural network designed to perform inference on data in any form, for example the Graph Neural Network (GNN) which performs inference on data described in graphs in a method coined by the present inventors as “Deep-CDR”.

In embodiments of a given target cancer antigen, the jointly trained model, such as Deep-CDR, may be tuned by further supervised training steps for the property of interest, such as the antigen of interest on 1000's of sequences, in house, or otherwise obtained, that are known to bind to the antigen of interest through an alternative method, such as CAR binding assays, ScFv binding, phage panning.

The final model is then used to expand and/or refine the in-silico generated CAR sequences from the method above (in as many iterations as required) by predicting CDR sequences that have improved binding to an antigen of interest.

Importantly the process can be run in an iterative fashion improving in each cycle until the desired level of functionality is obtained.

In a further embodiment, the output of the model may be refined by ranking and prioritisation methods.

Specific embodiments of such refinement methods are:
Embodiment 1: Select CARs that are predicted to bind favourable epitopes or with ideal cell-to-cell distance;
Embodiment 2: Select CARs that are predicted to express better;
Embodiment 3: Select CARs based on confidence score of the predictions;
Embodiment 4: Ranking: generate new CAR domains in isolation, randomly combine all these domains in silico and then use the CAR ML model to rank which of these combinations should be prioritised. e.g. new antibodies combined with 20 different hinges, combine all possibilities and filter/rank through the ML model (see “In silico CAR design to cover CAR space” section below).

### Test New CAR Sequences

In embodiments, new CAR sequences output in silico by virtue of the above methods may made/synthesised/assembled for different functional readouts.

If further refinement is required or desirable, the results may be input into an updated training set and the models re-defined before re-running. It is an important, although not essential, aspect of the present invention that further iterations of the model may be exploited to further refine the modelling and output.

### In Silico CAR Design to Cover CAR Space

The discussed methods above are amenable to generation of novel CARs from experimentally gathered datasets.

Disclosed herein is a further, tool for the de novo design and generation of CARs. In embodiments of this process known generative methods such as ARD (autoregressive diffusion) models may be trained on available sequence data for each of the CAR features (for example, see list below). These features will then be combined to make a large number of in-silico novel CAR candidate sequences.

In embodiments, the in-silico CAR sequence space can be generated in modular fashion by varying multiple components of the CAR-sequence individually or in combination.
Generating a sequence space of binding moieties (e.g. scFv, VHH, receptor-based)
Generating a sequence space of hinges
Generating a sequence space of linkers
Generating a sequence space of transmembrane domains
Generating a sequence space of intracellular domains (e.g. co-stimulatory, stimulatory, inhibitory, activatory, positive feedback loops, negative feedback loops)
Generating a CAR sequence space of combinations of the above domains of a CAR

The number of candidate CARs generated may be high. The number of candidate CARs in this set may be rationalised to align with processing capacity. For example, not all of the combinations would need to be generated and an algorithm, such as an exploration/exploitation trade off algorithm or other means of selection may be applied to most effectively explore the sequence space (machine learning may be used for selecting small representative sub-datasets).

The in-silico filtering of this large space of CAR sequences can be achieved through machine learning methods or other computational tools. Such tools can filter for trained observations and eliminate CARs with poor protein stability, tonic activation, aggregation propensity, non-accessible epitopes, non-optimal epitopes, low avidity, low specificity, low activation propensity, non-optimal T cell activation.

The CARs can be ranked based on predicted functionality and a subset can be selected. select a subset of these CARs. The selection may be made based on any suitable or relevant criteria. In embodiments, the selection may be made with a balance for exploration/exploitation (to maintain diversity and not test many of similar CARs).

These methods enable targeted generation of a small set of effective novel CAR-antigen complexes. Structural bioinformatics (e.g. AlphaFold™ multimer) may be used to render such methods more efficient and this will increase in time.

Important in each of the current approaches outlined herein is the integration of the high throughput functional CAR datasets, such as single-cell datasets (and other sequencing data used, such as next gen sequencing data of, for example a CDR or an scFv) with the machine learning or other computational strategy. The scope and scale of the data collected powers the machine learning methods and provides the annotation to build on. This includes the scope of the variation in all potential CAR features.

In all aspects of the present invention, an important consideration is breaking the embedding up to sub-sections to provide bespoke models and to take into account the engineered modular nature of the CAR proteins.

Also important is the construction of bespoke embedding methods. For example, CDR sequences are not well represented in publicly available embedding methods and need to be incorporated in a new model. Important (but not essential) for performance is mapping the embeddings to a lower dimensional latent space using autoencoders.

In embodiments, iterative cycles between experimental and machine learning data are an important feature for optimisation of results.

Relevant to the above, and all models specifically described herein, the specific choice of language model is not essential and is not a core feature of the module. Such methods are being improved all the time and these parts of the method will be updated as new models become the state of the art. Such changes can be made without altering the overall concept and structure of the process.

### EXAMPLES

### Example 1: Use of Computational CAR Models for Generative CAR Design

A CAR database was generated containing data relating to CAR sequences and their associated functional activity and/or properties, including their intended target, affinity, avidity, binding intensity and activation. The method of preparing the database was as described in International patent application no. PCT/GB2022/050158.

A large-scale protein language model (protBERT) was used to embed the CAR sequences. The model was pre-trained on CAR sequences, in this case from the Applicant's proprietary CAR sequence database, described immediately above although any suitable database would be suitable. This trains the model using full CAR sequences towards ‘CAR space’, i.e. CAR-like sequences. This training is by sequence only, representing a set of CARs having the property of being suitably expressed. Finally, fine tuning of the model was completed by using supervised training on the experimental sets of CAR sequences from the Applicant's proprietary CAR sequence database mapped to different relevant functional activities (e.g. binding, activation) (sequence plus properties). Fine tuning can be either regression (quantitative) or classification (qualitative, or a binary “yes” or “no”).

A further antibody language (ABlang) model was used for zero-shot prediction of alterations to the CDRs of the ScFv sequences. Fine-tuning of the model was performed using antibody-fragment sequence and binding data from the Applicant's proprietary CAR sequence database.

Using a CAR sequence targeting BCMA as a seed sequence, these generative models were used sequentially to design CAR sequences targeting BCMA that were predicted to have enhanced functional activity.

For the designed CAR sequences, predicted CAR sequences were synthesised (Integrated DNA Technologies) and amplified using Q5 DNA polymerase (NEB). The PCR products were cleaned and then ligated using BamHl and Mlul into a lentiviral vector. Subsequently, the plasmids were transformed into NEB DH5a chemically competent bacteria, followed by plasmid isolation. The CAR sequences were sequenced on an Oxford Nanopore Technology™ MinION™, using the amplicon sequencing kit.

In order to assess if the predicted CAR sequences were functionally expressed and recognizing BCMA, the CAR library against BCMA was used to produce lentiviral vector particles and was then transduced into primary human T cells. The resulting CAR-T cells were expanded for six more days after transduction and assessed for BCMA binding. BCMA-CAR-T cells were stained with BCMA-Fc fusion protein. In a second step, cells were stained with PE-conjugated anti-Fc and APC-conjugated anti-CD34 antibody to detect transduced cells. The expression of CARs on the cell surface was assessed (see FIG. 4). As shown, a number of the designed CARs shown significantly increased median fluorescence intensity than the lead candidate anti-BCMA CAR (highlighted with arrow), indicating enhanced CAR binding to the target, BCMA.

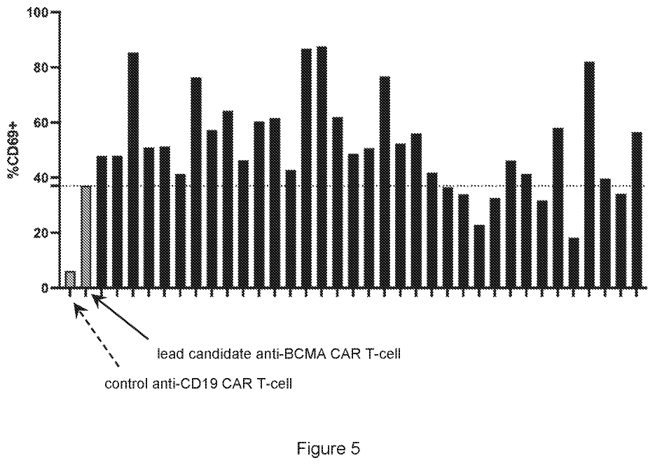

In order to assess the functional activity of the CAR-T constructs, anti-BCMA CAR-T cells were co-cultured with the BCMA-positive RPMI-8226 cell line for 24 hours. After 24 hours, cells were collected and stained with anti-CD3, anti-CD34, anti-CD69 and anti-CD25 antibodies. Cells were analysed by flow cytometry on a NovoCyte™ Flow Cytometer (Agilent™) (FIG. 5). The percentage of CD69 positive cells was measured for CAR-T cells grouped by their CAR-T construct. As shown, many of the predicted CAR sequences outperformed the lead candidate, demonstrating enhanced function.

Example 2: Use of Computational CAR Models for Generative CAR Design with CAR Space Expansion

A CAR database was generated containing data relating to CAR sequences and their associated functional activity, including their intended target, affinity, avidity, binding intensity and activation. The method of preparing the database was as described in International patent application no. PCT/GB2022/050158.

A GPT2 language model was pre-trained on in-house CAR sequences from the CAR sequence database, as described above.

The language model was used to generate de novo CAR sequences. Additionally, CAR regions were generated by masking regions of interest and using the language model to predict the masked region. From the resulting predicted CAR regions, random combinations were generated, yielding full CAR sequences.

Prioritisation of CAR sequences was achieved by first embedding the CAR sequences and subsequently using clustering techniques identify distinct clusters of sequences within the data. When prioritising CAR sequences, it was ensured that all major clusters were represented by at least one representative sequence in the final set of CAR sequences generated. This provides a useful trade off allowing the prioritisation of more promising CAR sequences, whilst ensuring exploration of diverse sequences in the final experimental CAR functional assays. Additionally, a confidence score was assigned using the language model, allowing in silico prediction of CAR function. CAR sequences that were predicted to have a high score were prioritised.

CAR sequences were synthetized and assembled as a pooled library of CARs in a lentiviral vector using Golden Gate assembly (NEB, BsmBI v2). Pooled libraries were sequenced on an Oxford Nanopore Technology™ MinlON™, using the amplicon sequencing kit.

A CAR-T library was generated using lentiviral vector transduction, two days after T cell activation. PBMCs were activated using TransAct (Miltenyi), in the presence of IL2, transduced with the CAR library and expanded for an additional six days.

At harvest, cells were activated with RPMI-8226 cell line for six hours. Subsequently CAR-T cells were selected using anti-CD34 microbeads (Miltenyi). Cells were washed and prepared for 5′ single cell sequencing analysis, following standard protocols (10×genomics).

CAR sequences and corresponding 10×barcodes were identified using long-read Oxford Nanopore Technology™ sequencing.

The resulting CAR T-cells were evaluated and optionally the results were added to the training set to further refine the model.

Example 3: Use of Computational CAR Models for Selection of CAR Regions or Components Thereof from a Screening Set

A CAR model was trained on the UniRef protein database and a CAR sequence database (such as the Applicant's proprietary CAR database) using a GPT2 (or optionally BERT or denoising/diffusion models).

Subsequently the model was fine-tuned on a set of CAR sequences with associated, experimentally derived labels/measurements (e.g cytotoxicity, cytokine production, affinity, cell phenotype, target-specificity). A list of CAR sequences was given as an input into the model and phenotype output (properties) was predicted. CAR sequence were ranked based on the output and a subset with highest (or lowest or combination of different outputs) was selected for experimental validation.

Although particular embodiments of the invention have been disclosed herein in detail, this has been done by way of example and for the purposes of illustration only. The aforementioned embodiments are not intended to be limiting with respect to the scope of the invention. It is contemplated by the inventors that various substitutions, alterations, and modifications may be made to the invention without departing from the spirit and scope of the invention.

1. A method for designing a chimeric antigen receptor (CAR), comprising:
a) defining a training set of one or more training CAR sequences, each of the one or more training CAR sequences in the training set encoding a training CAR, wherein each training CAR is associated with one or more properties;
b) defining one or more objectives, each of the one or more objectives defining a desired property of a CAR;
c) training a computational model using the training set of the one or more training CAR sequences to provide a trained computational model that outputs an approximation of the one or more properties of a CAR as a function of one or more features of a CAR sequence;
d) using the computational model to provide at least one output CAR sequence, wherein the at least one output CAR sequence is not in the training set, and wherein the at least one output CAR sequence is determined based on the one or more objectives, and wherein the at least one output CAR sequence encodes a CAR.

2. The method of claim 1, wherein the at least one output CAR sequence is determined based on predicted attainment of, or improvement in, one or more of the desired properties of a CAR as defined by the one or more objectives.

3. The method of claim 2, wherein the CAR encoded by the at least one output CAR sequence is prepared and evaluated so that one or more properties are associated with the CAR, to provide one or more output CARs with associated properties.

4. The method of claim 3, wherein the one or more output CARs with associated properties are added to the training set of training CAR sequences in step (a) and then method steps (b) to (d) are repeated.

5. The method of any one of claims 1 to 4, wherein the training set comprises one or more training CARs that have been chosen due to having functional activity against a pre-determined biological target.

6. The method of claim 5, wherein the one or more output CAR sequences encode a CAR that is predicted by the computational model to have improved or enhanced functional activity against the biological target.

7. The method of any one of claims 1 to 6, wherein the one or more properties are selected from the group consisting of: binding affinity; avidity; specificity; selectivity; cytotoxicity; cellular activation; intracellular signalling; tonic signalling; expression; surface expression; cytokine production; proliferation; exhaustion; serial killing ability of a cell in which the CAR is expressed; Kₒₙ/k_{off} rates of a cell in which the CAR is expressed; target binding location; epitope; and any combination thereof.

8. The method of claims 1 to 7, wherein the one or more features of the CAR are selected from the group consisting of: sequence; part of the sequence; 3D structure; primary structure of the CAR; secondary structure of the CAR; tertiary structure of the CAR; polarity; hydrophobicity; electrostatics; protein stability; thermal stability; vector distance between atoms or amino acids; linear distance between atoms or atoms; or any combination thereof.

9. The method of any one of claims 1 to 8, wherein the training set comprises a plurality of training CAR sequences.

10. The method of claim 9, wherein the minimum number of training CAR sequences in the training set is selected from the group consisting of: 100; 1000; 10,000; 100,000; 500,000; 1,000,000; 10,000,000; and 100,000,000.

11. The method of any one of claims 1 to 10, wherein each of the one or more training CAR sequences comprise an antigen binding domain sequence; a hinge domain sequence; a transmembrane domain sequence; and optionally an intracellular domain sequence.

12. The method of any one of claims 1 to 11, wherein each of the one or more output CAR sequences comprise an antigen binding domain sequence; a hinge domain sequence; a transmembrane domain sequence; and optionally an intracellular domain sequence.

13. The method of any one of claims 1 to 12, wherein the computational model is a machine learning inference model in which using the computational model comprises selection of the one or more output sequences from a screening set of one or more screening CAR sequences, wherein the one or more screening CAR sequences are not present in the training set.

14. The method of claim 13, wherein the screening set comprises one or more full CAR sequences comprising an antigen binding domain sequence, a hinge domain sequence, a transmembrane domain sequence and optionally an intracellular domain sequence.

15. The method of claim 13, wherein the screening set comprises sequences selected from the group consisting of: a set of one or more antigen binding domain sequences; a set of one or more hinge domain sequences; a set of one or more transmembrane domain sequences; and a set of one or more intracellular domain sequences; and a set of one or more sequences comprising a first domain of a CAR and one or more further domains of a CAR, wherein the first domain of a CAR is selected from the group consisting of: an antigen binding domain sequence, a hinge domain sequence, a transmembrane domain sequence and an intracellular domain sequence, and wherein the one or more further domains of a CAR are each independently selected from the group consisting of: antigen binding domain sequence, a hinge domain sequence, a transmembrane domain sequence and an intracellular domain sequence, wherein the first domain of a CAR is not the same domain as the second domain of a CAR, and wherein each member of the set may be recombined with appropriate remaining parts of a CAR to provide a full CAR sequence.

16. The method of any one of claims 13 to 15, wherein step (d) comprises:
d)i) providing the screening set of the one or more screening CAR sequences;
d)ii) selecting a subset of one or more CAR sequences from the screening set using the computational model, the subset of CAR sequences being determined according to selection and/or optimisation from the screening set based on the objectives.

17. The method of any one of claims 13 to 16, wherein one or more CAR sequences are embedded into vector representations.

18. The method of any one of claims 13 to 17, wherein state of the art (SOTA) protein embedding methods are used.

19. The method of claim 18, wherein the SOTA protein embedding method is Prot-T5.

20. The method of any one of claims 1 to 12, wherein the computational model is a deep learning method for generation of the one or more output sequences.

21. The method of claim 20, wherein step (d) comprises the step of:
d)i) generation of at least one output CAR sequence by the computational model based on the objectives, wherein the output CAR sequence is not in the training set, wherein the output CAR sequence encodes a CAR.

22. The method of claim 21, wherein the generation is de novo, or by development of a CAR sequence or part thereof that is provided to the trained computational model.

23. A method for training a computational model for designing a chimeric antigen receptor (CAR), comprising:
a) defining a training set of one or more training CAR sequences, each of the one or more training CAR sequences in the training set encoding a training CAR, wherein each training CAR is associated with one or more properties;
b) defining one or more objectives, each of the one or more objectives defining a desired property of a CAR;
c) training a computational model using the training set of the one or more training CAR sequences to provide a trained computational model that outputs an approximation of the one or more properties of a CAR as a function of one or more features of a CAR sequence.

24. The method of claim 23, wherein the training set comprises a plurality of training CAR sequences.

25. The method of claim 24, wherein the minimum number of training CAR sequences in the training set is selected from the group consisting of: 100; 1000; 10,000; 100,000; 500,000; 1,000,000; 10,000,000; and 100,000,000.

26. The method of claims 1 to 25, wherein the computational model is trained with additional training sets.

27. The method of claim 26, wherein the training of the model with additional training sets is sequential, before or after the step (c), or simultaneously with step (c).

28. The method of claim 27, wherein the additional training sets may be selected from the group consisting of: protein sequences; antibody sequences; CAR sequences; antigen binding domain sequences; hinge domain sequences; transmembrane domain sequences; intracellular domain sequences; and antigen sequences.

29. A trained computational model prepared by the method of any one of claims 23 to 28.

30. A CAR sequence output by the method of claims 1 to 22.

31. A CAR encoded by the CAR sequence of claim 30.

32. A cell expressing the CAR of claim 31.

33. The cell of claim 32, wherein the cell is an engineered immune cell.

34. The cell of claim 31 or claim 33, wherein the cell is a T-cell.

35. A non-transitory, computer-readable storage medium storing instructions thereon that when executed by a computer processor causes the computer processor to perform the method of claims 1 to 28.

36. A computing device, comprising:
an input arranged to receive:
i) data indicative of a training set of training CAR sequences, each training CAR sequence comprising an antigen binding domain; a hinge domain; a transmembrane domain; and an intracellular domain, wherein each training CAR sequence is associated with one or more properties; and
ii) data indicative of one or more objectives each of the one or more objectives defining a desired property;
a processor arranged to train, using the training set of training CARs, a computational model to provide an approximation of properties of a CAR as an function of one or more features of the CAR, and arranged to output at least one output CAR, which are not in the training set, and wherein the at least one output CAR sequence is determined based on the one or more objectives, and wherein the at least one output CAR sequence encodes a CAR; and
an output arranged to output the determined subset.

37. The computing device of claim 36, wherein the input is further arranged to receive:
iii) data indicative of a set of one or more CARs, each CAR comprising an antigen binding domain; a hinge domain; a transmembrane domain; and an intracellular domain,
wherein each CAR comprises one or more structural features.

38. The computing device of claim 36, wherein the input is further arranged to receive:
iii) data indicative of one or more sets of CAR domain sequences, each set encoding sequences that encode separate CAR domains selected from the group consisting of:
an antigen binding domain; a hinge domain; a transmembrane domain; and an intracellular domain, or combinations thereof, wherein each CAR domain comprises one or more features.

39. The computing device of claim 36, wherein the computational model generates at least one output CAR sequence determined based on the one or more objectives.
